(19) United States
(12) Patent Application Publication
(10) Pub. No.: US 2021/0128932 A1
(43) Pub. Date: May 6, 2021

(54) CHRONOTHERAPEUTIC TREATMENT PROFILING

(71) Applicant: Emblation Limited, Alloa (GB)

(72) Inventors: Eamon McErlean, Alloa (GB); Gary Beale, Edinburgh (GB)

(73) Assignee: Emblation Limited, Alloa (GB)

(21) Appl. No.: 16/974,000

(22) PCT Filed: Jun. 17, 2019
(86) PCT No.: PCT/GB2019/051681

§ 371 Date: Dec. 10, 2020

(30) Foreign Application Priority Data
Jun. 15, 2018 (GB) 1809890.5

(51) Int. Cl. A61N 5/02; A61B 5/318; A61B 5/0205; A61B 5/369; A61N 1/36; A61N 5/10; G01N 33/50

(52) U.S. Cl. CPC A61N 5/025; A61B 5/318; A61B 5/0205; A61B 5/021; A61N 1/36014; A61N 5/10; G01N 33/5091; A61B 5/369

(57) ABSTRACT

An electromagnetic energy delivery system is configured to deliver energy in specific timing arrangements to improve an immune response. The electromagnetic energy delivery system may comprise a controlled electromagnetic energy source and an electromagnetic energy applicator, wherein the controlled electromagnetic energy source is configured to emit electromagnetic energy according to the specific timing arrangements as defined by a treatment profile which is configured so that the emitted electromagnetic energy stimulates and/or inhibits an immune response in a host when the emitted electromagnetic energy is delivered to the host via the electromagnetic energy applicator. A method of electromagnetic energy delivery,comprises delivering energy in specific timing arrangements to improve an immune response.

### FIELD

An electromagnetic energy delivery method is described comprising delivering electromagnetic energy in specific timing arrangements to improve an immune response. The method may utilise a variety of temporal treatment variables to facilitate an immune response by the application of electromagnetic energy to a host.

An electromagnetic energy delivery system is described that delivers electromagnetic energy in specific timing arrangements to improve an immune response.

### BACKGROUND

Methods and systems are known for ablating tissue of a host using electromagnetic energy. Such systems generally deliver electromagnetic energy to the host from an energy generator, via a connecting cable, to a radiating applicator that transfers the energy into the tissue of host. In these applicators, the radiating element is surrounded by tissue or is placed in contact with the tissue. For such systems, the typical standard practice is to deliver energy for a treatment lasting typically anywhere from 1-20 minutes to raise the temperature of tissue greater than 43-45 to 60, 70+° C. and beyond such that necrosis occurs within the desired ablation zone. The energy may be delivered to have an amplitude or pulse width-modulated duty cycle to ensure the required level of energy is maintained or controlled for the duration of the energy release.

These standard types of electromagnetic generator systems are designed to destroy diseases or unwanted tissue

### SUMMARY

It should be understood that one or more features of any one of the following aspects may be provided in any combination with one or more features of any of the other aspects.

According to an aspect of the present disclosure there is provided an electromagnetic energy delivery system that delivers energy in specific timing arrangements to improve an immune response.

Various beneficial methods and delivery profiles are described herein relating to delivery of electromagnetic energy such as microwave energy to promote or inhibit an immune reaction.

Combinations of various pulse regimes, modulation schemes, treatment duration, treatment intervals, immunogical measurement and nervous system feedback methods are described relating to the application of electromagnetic energy to influence an immune response.

The methods described herein are designed to deliver treatments in a fundamentally immune responsive way, in concert with the adaptive or innate biological immune systems. Immune response may be classed for example as: upregulation or down regulation of signalling; suppression or promotion of cell type growth, induction of apoptosis, modulation of cellular membrane.

In contrast with traditional ablative treatments, a key aspect of immune response optimised treatments is the synergistic control of energy application in the temporal micro-scale, the temporal macro-scale and in other biological time-scales that correlate with optimum immune response in diseased or abnormal tissues. A combination of these requirements or elements thereof may be beneficial in the overall treatment profile to ensure such responses.

Without wishing to be bound by theory, in terms of micro-scale (seconds), for particular neurophysiological events the throbbing pain rate (1 Hz or less) may closely approximate the rate of the rhythmic modulation of alpha wave power as opposed to the arterial pulse. Pulsed electromagnetic fields (e.g. 5 Hz) may down regulate inflammatory pathway markers in murine macrophages. The use of pulsed electromagnetic fields may activate mitogen-activated protein (MAP) kinase, initiating cell responses that lead to cell proliferation.

In terms of macro-scale (hours/days/weeks) it is postulated that the immune system behaves in a cyclic manner, for example a cycle may last 12 to 14 days depending upon the individual and that treatments targeted to this cycle could optimise their effectiveness. Acute exposure to microwave energy for a brief duration may activate macrophages that are significantly more viricidal, and become activated as early as 6 hours after exposure and remain activated for up to 12 days. In the wound healing cascade, the inflammation and macrophage stages may last from 2 to 4 weeks as part of the healing phase.

Interruption of the cycle with further treatment dose may activate protective responses that could potentially down regulate immune activation. Likewise, protracted delay between treatments may be detrimental to overall healing or duration if this corresponds with a period where the immune system is less active i.e. the response is not of as great a magnitude in an optimised regime

Artificially stimulating the vagus nerve may control the activation of circulating immune cells and conversely with diminished vagus nerve signals providing an inhibitory influence on cytokine production.

Microwaves may directly interact with cellular membranes, for example to mimic the effects of ligands binding with specific receptors producing direct microwave related (non-thermal) effects. An intact inflammatory response may be important in maintaining a normal immunological defence mechanism. In some conditions where the immune system is compromised (viral infection) an increase in inflammatory response may be required. In some other conditions, a reduction in inflammatory response may be required, e.g. vascular occlusion.

Such nervous signalling systems may be available for use, e.g. to monitor and influence to promote or to reduce immune actions as required.

The electromagnetic energy delivery system may comprise a controlled electromagnetic energy source and an electromagnetic energy applicator, wherein the controlled electromagnetic energy source is configured to emit electromagnetic energy according to the specific timing arrangements as defined by a treatment profile which is configured so that the emitted electromagnetic energy stimulates and/or inhibits an immune response in a host when the emitted electromagnetic energy is delivered to the host via the electromagnetic energy applicator.

The controlled electromagnetic energy source may be configured to emit electromagnetic energy according to a treatment profile which is configured so that the emitted electromagnetic energy stimulates and/or inhibits an immune response in the host without causing necrosis of cells or tissue of the host when the emitted electromagnetic energy is delivered to the host via the electromagnetic energy applicator.

The controlled electromagnetic energy source may be configured to emit electromagnetic energy according to a treatment profile which is configured so that the emitted electromagnetic energy heats cells or tissue of the host when the emitted electromagnetic energy is delivered to the host via the electromagnetic energy applicator.

The controlled electromagnetic energy source may be configured to emit electromagnetic energy according to a treatment profile which is configured so that the emitted electromagnetic energy heats cells or tissue of the host without ablating the cells or tissue of the host when the emitted electromagnetic energy is delivered to the host via the electromagnetic energy applicator.

The treatment profile may define one or more delivery periods and the controlled electromagnetic energy source is configured to only emit electromagnetic energy only during each delivery period.

The treatment profile may define one or more series of pulses, each pulse having a duration equal to the delivery period.

Each delivery period may be one thousandth of a second, one second, two seconds, two to three seconds, or any time duration up to twenty seconds or up to one or two or ten minutes.

The treatment profile may define one, five to ten, up to one hundred or up to one thousand delivery periods.

The treatment profile may define a plurality of delivery periods configured so that the emitted electromagnetic energy is pulsed at a rate of 5 Hz.

The controlled electromagnetic energy source may be configured to emit electromagnetic energy according to a treatment profile which is configured so that the emitted electromagnetic energy is emitted with a power in the range 1-50 W, such as 8 W-10 W, 2 W-5 W or 3 W-8 W.

The controlled electromagnetic energy source may be configured to emit electromagnetic energy according to a treatment profile which is configured so that the emitted electromagnetic energy is emitted in a series of five pulses, each pulse having a two second delivery period.

The controlled electromagnetic energy source may be configured to emit electromagnetic energy at a microwave frequency.

The controlled electromagnetic energy source may be configured to emit electromagnetic energy in a frequency range of 1-300 GHz.

The controlled electromagnetic energy source may be configured to emit electromagnetic energy according to a treatment profile which is configured so that the emitted electromagnetic energy is amplitude modulated.

The controlled electromagnetic energy source may be configured to emit electromagnetic energy according to a treatment profile which is configured so that the emitted electromagnetic energy is amplitude modulated at a frequency in the range 1-100 KHz.

The controlled electromagnetic energy source may be configured to emit electromagnetic energy according to a treatment profile which is configured so that the emitted electromagnetic energy is modulated according to a pulse width modulation (PWM) or an on/off keying (OOK) modulation scheme.

The controlled electromagnetic energy source may be configured to emit electromagnetic energy according to a treatment profile which is configured so that the emitted electromagnetic energy is frequency modulated.

The controlled electromagnetic energy source may be configured to emit electromagnetic energy according to a treatment profile which is configured so that the emitted electromagnetic energy is frequency modulated at a frequency in the range 1-100 KHz.

The controlled electromagnetic energy source may be configured to receive one or more signals from one of more physiological parameter sensors and to adapt the treatment profile according to the one or more received signals.

The controlled electromagnetic energy source may be configured to receive a PQRST heart rhythm signal from an ECG sensor and to adapt the treatment profile according to the PQRST heart rhythm cycle signal, for example by dynamically synchronising the treatment profile to correspond with a particular point in the PQRST heart rhythm cycle.

The controlled electromagnetic energy source may be configured to receive a neural oscillation signal from an EEG sensor and to adapt the treatment profile according to the neural oscillation signal, for example by dynamically synchronising the treatment profile to correspond with the neural oscillation signal.

The controlled electromagnetic energy source may be configured to receive a blood pressure signal from a blood pressure sensor and to adapt the treatment profile according to the blood pressure signal, for example by dynamically synchronising the treatment profile to correspond with the blood pressure signal.

The electromagnetic energy delivery system may be configured for use with specific treatment protocols (e.g. 10 W, 2 second, 5× repeats).

The electromagnetic energy delivery system may be configured for use with specific 4 week treatments.

The electromagnetic energy delivery system may be configured for use with a minimum 12 week follow up.

According to an aspect of the present disclosure there is provided an electromagnetic energy delivery system that combines heating and biological stimulation, e.g. dielectrophoresis.

The electromagnetic energy delivery system may be configured for use with high frequency electromagnetic energy 1-300 GHZ containing amplitude 1-100 KHz.

The electromagnetic energy delivery system may be configured for use with high frequency electromagnetic energy 1-300 GHZ containing amplitude and frequency modulation 1-100 KHz.

The electromagnetic energy delivery system may be dynamically synchronised to correspond with a particular points in the PQRST heart rhythm cycle.

The electromagnetic energy delivery system may be dynamically synchronised to correspond with neural oscillations.

The electromagnetic energy delivery system may be configured to monitor physiological parameters and dynamically adapt the energy delivered according to the physiological parameters.

The electromagnetic energy delivery system may be configured to adapt the energy delivered on a timescale of seconds, for example on a timescale of 5 seconds or less, 2 seconds or less, or 1 second or less, or on a timescale of a fraction of a second.

According to an aspect of the present disclosure there is provided an electromagnetic energy delivery system that delivers energy in specific timing arrangements in conjunction with immune cycle mapped to optimise the immune response.

According to an aspect of the present disclosure there is provided an electromagnetic energy delivery system that forms a treatment method in conjunction with other therapies (radiotherapy, chemotherapy, immunotherapy and traditional pharmacological therapies) where the temporal delivery is optimised from measurements from the host system.

According to an aspect of the present disclosure there is provided a method of electromagnetic energy delivery comprising delivering energy in specific timing arrangements to improve an immune response.

The method of electromagnetic energy delivery may comprise emitting electromagnetic energy according to the specific timing arrangements as defined by a treatment profile and delivering the emitted electromagnetic energy to a host, wherein the treatment profile is configured so that the emitted electromagnetic energy stimulates and/or inhibits the immune response in the host when the emitted electromagnetic energy is delivered to the host.

The method of electromagnetic energy delivery may comprise emitting electromagnetic energy according to a treatment profile which is configured so that the emitted electromagnetic energy stimulates and/or inhibits an immune response in the host without causing necrosis of cells or tissue of the host when the emitted electromagnetic energy is delivered to the host.

The method of electromagnetic energy delivery may comprise emitting electromagnetic energy according to a treatment profile which is configured so that the emitted electromagnetic energy heats cells or tissue of the host when the emitted electromagnetic energy is delivered to the host.

The method of electromagnetic energy delivery may comprise emitting electromagnetic energy according to a treatment profile which is configured so that the emitted electromagnetic energy heats cells or tissue of the host without ablating the cells or tissue of the host when the emitted electromagnetic energy is delivered to the host.

The treatment profile may define one or more delivery periods and the method comprises only emitting electromagnetic energy during each delivery period.

The treatment profile may define one or more series of pulses, each pulse having a duration equal to the delivery period.

Each delivery period may be one thousandth of a second, one second, two seconds, two to three seconds, or any time duration up to twenty seconds or up to one or two or ten minutes.

The treatment profile may define one, five to ten, up to one hundred or up to one thousand delivery periods.

The treatment profile may define a plurality of delivery periods configured so that the emitted electromagnetic energy is pulsed at a rate of 5 Hz.

The treatment profile may define a schedule of treatments, each treatment compising at least one delivery period, and each treatment being separated by an interval of one or more weeks, for example four weeks.

The treatment profile may define one or more further delivery periods after a follow-up interval of at least 12 weeks.

The method may comprise emitting electromagnetic energy according to a treatment profile which is configured so that the emitted electromagnetic energy is emitted with a power in the range 1-50 W, such as 8 W-10 W, 2 W-5 W or 3 W-8 W.

The method may comprise emitting electromagnetic energy according to a treatment profile which is configured so that the emitted electromagnetic energy is emitted in a series of five pulses, each pulse having a two second delivery period.

The method may comprise emitting electromagnetic energy at a microwave frequency.

The method may comprise emitting electromagnetic energy at a frequency in the range 1-300 GHz.

The method may comprise emitting electromagnetic energy according to a treatment profile which is configured so that the emitted electromagnetic energy is amplitude modulated.

The method may comprise emitting electromagnetic energy according to a treatment profile which is configured so that the emitted electromagnetic energy is amplitude modulated at a frequency in the range 1-100 KHz.

The method may comprise emitting electromagnetic energy according to a treatment profile which is configured so that the emitted electromagnetic energy is frequency modulated.

The method may comprise emitting electromagnetic energy according to a treatment profile which is configured so that the emitted electromagnetic energy is frequency modulated at a frequency in the range 1-100 KHz.

The method may comprise receiving one or more signals from one of more physiological parameter sensors and adapting the treatment profile according to the one or more received signals.

The method may comprise receiving a PQRST heart rhythm signal from an ECG sensor and adapting the treatment profile according to the PQRST heart rhythm cycle signal, for example by dynamically synchronising the treatment profile to correspond with a particular point in the PQRST heart rhythm cycle.

The method may comprise receiving a neural oscillation signal from an EEG sensor and adapting the treatment profile according to the neural oscillation signal, for example by dynamically synchronising the treatment profile to correspond with the neural oscillation signal.

The method may comprise receiving a blood pressure signal from a blood pressure sensor and adapting the treatment profile according to the blood pressure signal, for example by dynamically synchronising the treatment profile to correspond with the blood pressure signal.

The method may comprise adapting the treatment profile according to a measurement representative of an immune cycle of the host.

The measurement representative of the immune cycle of the host may comprise at least one of measurement of C-reactive protein (CRP) levels in the blood, T-regulatory cells (low state) and T-effector cells (high states).

The method may comprise administering one or more other therapies to the host, before, during and/or after emitting electromagnetic energy according to the treatment profile and delivering the electromagnetic energy to the host.

The one or more other therapies may comprise at least one of radiotherapy, chemotherapy, immunotherapy and traditional pharmacological therapies.

The one or more other therapies may comprise transcutaneous electrical nerve stimulation (TENS) and/or frequency rhythmic electrical modulation systems (FREMS).

According to an aspect of the present disclosure there is provided a treatment profile for use with any of the systems or methods of electromagnetic energy delivery described above.

Electromagnetic energy may be delivered to the host in specific temporal dosage profiles or protocols to elicit and/or optimise various immunological responses. These protocols encompass temporal dosage for the initial, treatment phase and healing/immune cycle phases to promote optimum immune responses in the tissue by the host immune systems.

Electromagnetic energy may be delivered to the host to elicit immune responses by delivering energy to biological tissues for ablative or non-ablative purposes.

Applied energy may be in the form of a continuous oscillating electromagnetic wave (CW) at a fixed frequency or modulated (variable frequency). The frequency could range from 1 MHz to 300 GHz but preferentially could be in the microwave range from 0.9 GHz to 16 GHz.

Pulse regimes include amplitude control of signal energy (AM pulsing) and pulse width modulation control (PWM) and on/off keying (OOK).

Modulation schemes include pulse modulation rates (1- 10 kHz) or frequency modulation rate (1-100 kHz)

Treatment durations may be single shot or multiple shot or continuous energy dose for a treatment session. A single shot could be one thousandth of a second, one second, two seconds, or any time duration up to twenty seconds or up to one or two or ten minutes followed by cessation of energy delivery. Preferentially a single shot could be two-three seconds, at a power level that does not result in ablative temperatures.

A multiple shot is a repeat of a single shot as described above for a number of treatment doses from one to one hundred or one thousand doses in a treatment session. Preferentially multiple shots could be five-ten times during a treatment dose.

A continuous dose may be a fixed level of energy or a modulated level of energy during a treatment session. This continuous energy delivery could be pulsed modulated one or five or fifty times a second during the ongoing treatment session. Preferentially continuous energy delivery could be pulsed modulated could be five times per second (5 Hz)

Treatment intervals describe the period between treatments which could be twice a week, once a week, once every 2 weeks, once every 4 weeks, monthly, fourteen days or other multi-day time period. Preferentially a four week interval between treatments would be optimal to address some viral conditions.

Immunogical measurement describes determination of immune system parameters to measure the effectiveness of the immune system (immune cycle mapping) e.g. C-reactive protein (CRP) levels in the blood, T-regulatory cells (low state) or T-effector cells (high states) to be used to determine the optimal time for treatment (immune cycle synchronisation) to elicit either the strongest immune response or to reduce the treatment dose to maintain an effective response.

Nervous system feedback methods may include electro-physical monitoring the heartbeat, neural oscillations (Alpha 8-13 Hz, Beta 16-31 Hz, Delta 0.5-3 Hz, Theta 4-7 Hz, Mu 7.5-12.5 Hz, SMR 12.5-15.5 Hz, Gamma 32-100 Hz) monitoring of afferent nerves (e.g. vagus nerve), and/or stimulation of efferent nerves, monitoring and stimulation of central nervous system or peripheral nervous system or synchronisation of applied energy to correlate with a particular natural oscillation to either be in or out of phase or to lead or lag, for example between cardiac sinus rhythms, in synchronisation with alpha waves or in proportional synchronisation with any natural oscillation. Preferentially synchronisation with Alpha oscillations would be desired.

In an aspect, which may be provided independently, there is provided an electromagnetic energy delivery system or method that combines heating and biological stimulation, for example dielectrophoresis.

The or each system may be configured to deliver high frequency electromagnetic energy, for example electromagnetic energy having a frequency in the range 1-300 GHZ and/or having amplitude modulation in a range 1-100 KHz

The or each system may be configured to deliver high frequency electromagnetic energy high frequency electromagnetic energy, for example electromagnetic energy having a frequency in the range 1- 300 GHZ and/or having containing amplitude and frequency modulation for example in a range 1-100 KHz

The or each system may be configured to deliver specific treatment protocols, for example application of electromagnetic energy with a selected power level and/or selected duration and/or selected number of repetitions (e.g 10 W 2 second 5× repeats)

There may be provided a method provided specific 4 week treatments using said system.

There may be provided a minimum 12 week follow up

In a further aspect, which may be provided independently, there is provided an electromagnetic energy delivery system or method that is dynamically synchronised to correspond with a particular points in a heart rhythm cycle, for example the PQRST heart rhythm cycle.

In a further aspect, which may be provided independently, there is provided an electromagnetic energy delivery system or method that is dynamically synchronised to correspond with neural oscillations

In a further aspect, which may be provided independently, there is provided an electromagnetic energy delivery system or method that monitors physiological parameters and dynamically adapts the energy delivery and/or energy delivery system in response to said monitoring.

The adaption may be in seconds or fractions of a second.

In a further aspect, which may be provided independently, there is provided an electromagnetic energy delivery system or method that delivers energy in specific timing arrangements in conjunction with immune cycle mapped to optimise the immune response

In a further aspect, which may be provided independently, there is provided an electromagnetic energy delivery system or method that forms a treatment method in conjunction with other therapies (radiotherapy, chemotherapy, immunotherapy and traditional pharmacological therapies) where the temporal delivery is optimised from measurements from the host system.

Features of one aspect may be provided in combination with features of any other aspect. Any one of system, method or apparatus features may be provided as any other of system, method or apparatus features.

According to embodiments, feature(s) of any one of the claims may be combined with feature(s) of any one or more other of the claims, regardless of the dependency of the claims.

### BRIEF DESCRIPTION OF DRAWINGS

Electromagnetic treatment systems and methods will now be described by way of non-limiting example only with reference to the accompanying drawings of which:

FIG. 1 is a diagrammatic illustration of an electromagnetic energy delivery system for stimulating and/or inhibiting an immune response in a host;

FIG. 2 is a diagrammatic illustration of a first electromagnetic energy treatment profile according to which the electromagnetic energy delivery system of FIG. 1 may be used, the electromagnetic energy treatment profile including periodic energy delivery intervals containing continuous-wave energy;

FIG. 3 is a diagrammatic illustration of a second electromagnetic energy treatment profile according to which the electromagnetic energy delivery system of FIG. 1 may be used, the electromagnetic energy treatment profile including periodic energy delivery intervals containing amplitude modulated energy;

FIG. 4 is a diagrammatic illustration of a third electromagnetic energy treatment profile according to which the electromagnetic energy delivery system of FIG. 1 may be used, the electromagnetic energy treatment profile including periodic energy delivery intervals of amplitude modulated energy and frequency modulated energy;

FIG. 5 is a diagrammatic illustration of a fourth electromagnetic energy treatment profile according to which the electromagnetic energy delivery system of FIG. 1 may be used, the electromagnetic energy treatment profile including periodic energy delivery intervals of continuous-wave energy and/or amplitude modulated energy and/or frequency modulated energy;

FIG. 6 is a diagrammatic illustration of additional electromagnetic energy treatment profiles according to which the electromagnetic energy delivery system of FIG. 1 may be used, the electromagnetic energy treatment profiles being arranged to dynamically occur within specific points corresponding with a measured sinus rhythm;

FIG. 7 is a diagrammatic illustration of typical neural oscillations;

FIG. 8 is a diagrammatic illustration of a yet further treatment profile according to which the electromagnetic energy delivery system of FIG. 1 may be used, the electromagnetic energy treatment profile including multiple treatment episodes and intervals spaced across a period of time; and

FIG. 9 is a diagrammatic illustration of part of an immune system cycle.

### DETAILED DESCRIPTION OF THE DRAWINGS

Electromagnetic treatment systems and methods will now be described purely by way of example. One of ordinary skill in the art will understand that modifications of the details of any of the electromagnetic treatment systems and methods described below may be made without departing from the scope of the invention as defined by the appended claims.

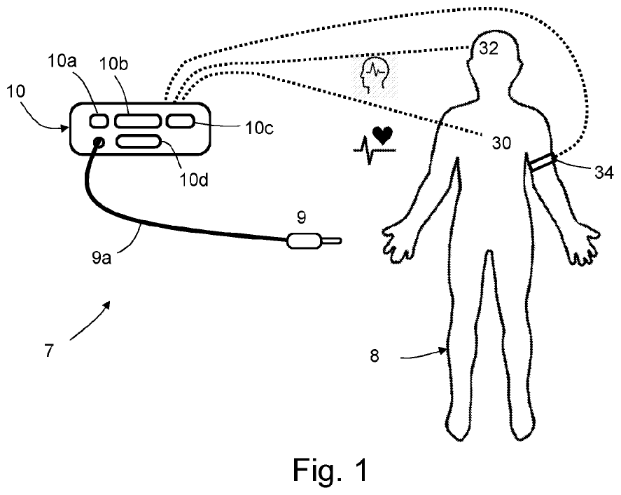

Referring initially to FIG. 1 there is shown an electromagnetic energy delivery system generally designated 7 for stimulating and/or inhibiting an immune response in a host or patient generally designated 8.

The system 7 includes a controlled electromagnetic energy source generally designated 10, an electromagnetic energy applicator 9 including one or more antennas for radiating and/or applying electromagnetic energy to the host 8, and a cable 9a for transmitting electromagnetic energy from the controlled electromagnetic energy source 10 to the electromagnetic energy applicator 9.

The controlled electromagnetic energy source 10 includes an electromagnetic energy source 10a, a processing resource 10b, a memory 10c, and a user interface 10d. The memory 10c contains instructions which, when executed by the processing resource 10b, cause the processing resource 10b to control the electromagnetic energy source 10a to emit electromagnetic energy according to one or more treatment profiles. The one or more treatment profiles may, for example, be stored in the memory 10c. Additionally or alternatively, the one or more treatment profiles may be manually input via the user interface 10d.

The cable 9a includes, or takes the form of, a waveguide for transmitting the electromagnetic energy emitted by the electromagnetic energy source 10a to the one or more antennas of the electromagnetic energy applicator 9. The cable 9a may include, or take the form of, a co-axial cable. The cable 9a may be flexible or rigid.

In use, the electromagnetic energy applicator 9 is held adjacent to, and/or in contact with, the host 8 and the processing resource 10b controls the electromagnetic energy source 10a to emit electromagnetic energy according to one or more of the treatment profiles for delivery of electromagnetic energy to the host 8 according to the one or more of the treatment profiles via the cable 9a and the electromagnetic energy applicator 9. In an exemplary embodiment, the controlled electromagnetic energy source 10 may be configured for applying microwave energy to the host 8 and the electromagnetic energy applicator 9 may be a microwave applicator. In such an embodiment, the electromagnetic energy source 10a may be configured to emit microwave energy and the cable 9a may be configured to transmit the emitted microwave energy to the one or more antennas of the microwave applicator 9.

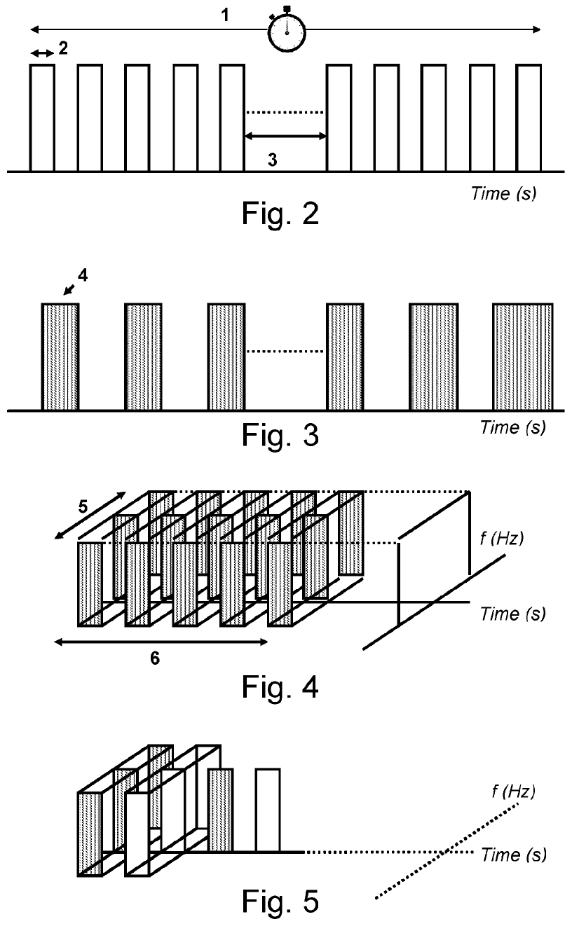

A first treatment profile is illustrated in FIG. 2. In this treatment profile there is a time duration 1 which represents the overall treatment time. This may be in seconds, minutes or hours and specifically may be one to thirty minutes. Within this time duration 1 a number of energy delivery periods 2 exist. These periods may be proportions of one second to five seconds, ten seconds, twenty seconds or any other period that is a proportion of the time duration 1.

A fixed or variable number of these energy delivery periods may be delivered and may include a treatment interval 3. The interval can be between each energy delivery period or could be a longer interval between a series of energy delivery periods. An example of this would be a microwave treatment system that delivers ten Watts of energy for a period of two seconds and each energy delivery period is repeated five times, with this cycle being repeated for up to fifteen minutes.

A second treatment profile is illustrated in FIG. 3. This treatment profile represents a number of energy delivery periods containing modulated energy 4, advantageously the energy delivery period can be dynamically altered to suit treatment requirements. The modulation may be pulse width modulation or amplitude modulation typically from 1 to 10 kHz.

A third treatment profile is illustrated in FIG. 4 in which the signals are frequency modulated 5 and delivered temporally 6; in this case a lower frequency signal is superimposed on a high frequency signal. This can be achieved as amplitude or frequency modulation or superposition of both resulting in the following:
a. Carrier, e.g. 8 GHz,
b. AM PWM modulation, e.g. 1-10 KHz,
c. Frequency modulation of 8 GHz carrier, e.g. 100-200 MHz/1-100 KHz

A fourth treatment profile is illustrated in FIG. 5 in which the foregoing modulation schemes are dynamically applied to be frequency modulated/amplitude modulated, frequency modulated continuous wave, fixed frequency/amplitude modulated or fixed frequency. Advantages of an interval pulsing scheme are that the temperature rise can be controlled to be within a therapeutic thermal window. Exceeding the therapeutic window can be detrimental to the tissues and cause necrosis though heat damage. When interspersing the energy delivery, the heat dissipation through natural perfusion allows excess heat to be transported away from the treatment zone. This is of consideration when optimal immune responses are found to be a function of exposure time to the electromagnetic field. Additionally, the rate of temperature rise at given optimal frequencies and amplitudes may exceed the therapeutic thermal window thus control over the rate of energy delivery is also an advantage.

This modulation may be chosen to effect a biological process in addition to a heating process, e.g. a modulated dielectrophoretic effect (AC electro-osmosis and dielectrophoresis) where non-uniform electromagnetic fields as a result of modulation are used to disrupt cellular membranes (cellular elution and ionic transportation via membrane proteins). The ion channels in cells experiencing dielectrophoresis are limited in their ability to conduct in both directions. As this mechanism can act directionally it can be utilised to effectively demodulate carrier signals as the ions behave akin to an electronic diode. Some ion channels intrinsically act directionally and behave as diodes in the absence of field gradients. The modulation of frequency can be utilised to selectively cover a variety of ion channels and to take advantage of resonant effects.

This disruption in cellular signalling can be utilised to promote cell death via apoptosis as opposed to necrosis. This differs from standard irreversible electroporation where a very strong electrical field of more than 0.5 V/nm is applied in nanosecond intervals to motate water molecules forming pores in the cellular membrane. Irreversible electroporation has the disadvantage of inducing muscle contractions requiring neuromuscular blockade.

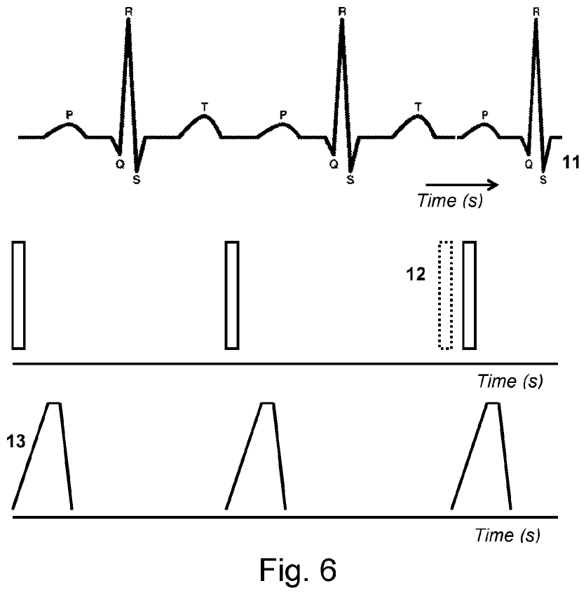

In addition, to avoid or utilise electrocardial involvement in electromagnetic treatments, energy delivery periods can be dynamically allocated 12 or synchronised to correspond with a particular point in a PQRST heart rhythm cycle 11 as illustrated in FIG. 6. In addition, the energy delivery periods 13 can be ramped, delayed, decayed or modified to create bespoke treatment profiles.

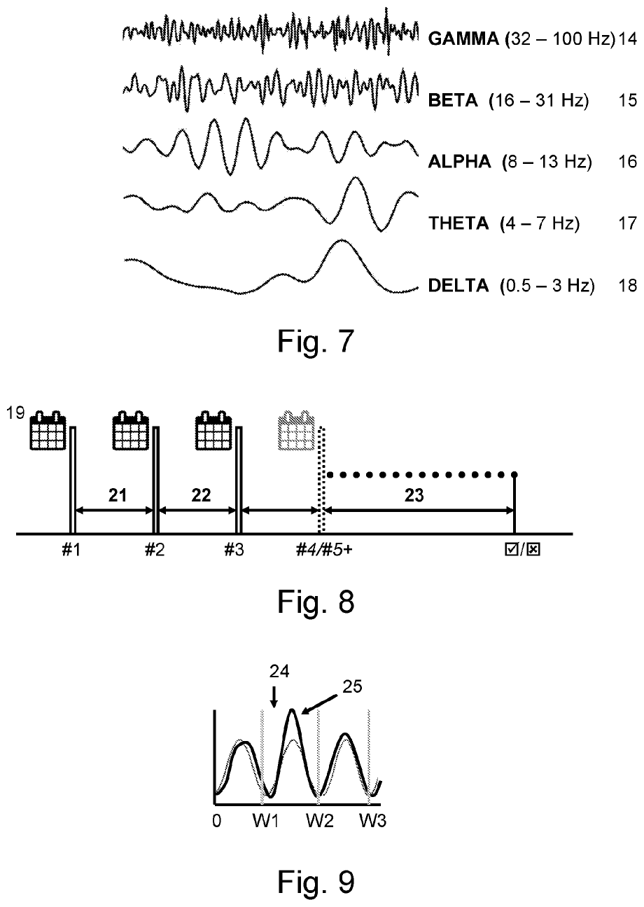

Knowledge of the physiology can also be used to tailor the treatment to meet particular requirements. For example, referring back to FIG. 1, the controlled microwave energy source 10may optionally receive an input from at least one of: an electrocardiogram (ECG) sensor 30, an electroencephalogram (EEG) sensor 32, a blood pressure sensor 34, and other physiological inputs (not shown) which may be relevant to a treatment. An example of neural waveforms available from an electroencephalogram sensor 32 is described with reference to FIG. 7. These waveforms represent different states of neural activity and use of these measurements may be made to synchronise treatment delivery. For example, the delivery period could synchronise with Alpha waves to stimulate a pseudo-throbbing response. This feedback response can be used to stimulate the immune system in dealing with conditions that may be masked from the immune system, e.g., HPV, Melanoma, carcinoma, viral lesions.

Primary electromagnetic energy delivery could also be combined with a secondary adjunctive energy delivery such as transcutaneous electrical nerve stimulation (TENS) or Frequency rhythmic electrical modulation systems (FREMS) to advantageously stimulate the immune system. Another adjunctive combination method include radiotherapy, chemotherapy, immunotherapy and traditional pharmacological therapies. The sequencing of these other therapies with the temporal electromagnetic treatment may also be derived from diagnostic physiological feedback from the patient.

A further advantageous aspect to the temporal delivery of treatments has been determined via post market surveillance of treatment efficacy data relating to viral lesions. FIG. 8 shows a treatment profile in which a number of treatment sessions 19 are delivered spaced apart by long time intervals. The optimal treatment delivery schedule spacing 21, 22 for each treatment #1, #2, #3 is one month (4 weeks) and the optimum review period 23 after cessation of treatment is 12 weeks. It is understood that this regimen promotes the optimum immune involvement. Reducing the schedule spacing interrupts the natural immune cycle and results in a less efficacious outcome. This optimum treatment profile with 12 week review cycle can raise the 76% efficacy reported to over 90% (unreported).

Knowledge of the immune cycle can be used to further boost the efficacy or alternatively reduce the number of treatments required. An example of a measure representative of the immune cycle is illustrated in FIG. 9. In this illustration, a measurement of C-reactive protein (CRP) levels in the blood, T-regulatory cells (low state) or T-effector cells (high states) can be used to determine the optimal time for treatment (immune cycle synchronisation) to elicit either the strongest immune response or to reduce the treatment dose required to produce an effective response. In FIG. 9, the largest peak 25 in the measure representative of the immune cycle occurs once every 2-3 weeks. The onset 24 of the growth in the measure representative of the immune cycle which precedes this peak 25 (i.e. the time 24 when the rise rate in the measure representative of the immune cycle increases towards the peak 25) can be utilised to create a stronger immune response, or alternatively to reduce the treatment levels required to achieve the same immune response. This may also correlate with other natural cycles such as resting heart rate (RHR). The optimal window of opportunity for administering a treatment occurs between the onset 24 of the growth in the measure representative of the immune cycle and the largest peak 25 in the measure representative of the immune cycle 25.

One of ordinary skill in the art will understand that the periodicity used for the temporal electromagnetic treatment may vary from the feedback derived from measurements taken by sampling and or tracking the levels of a marker, or in realtime with the use of EEG, ECG realtime measurements.

1. An electromagnetic energy delivery system that delivers energy in specific timing arrangements to improve an immune response.

2. The electromagnetic energy delivery system as claimed in claim 1, comprising:
a controlled electromagnetic energy source; and
an electromagnetic energy applicator,
wherein the controlled electromagnetic energy source is configured to emit electromagnetic energy according to the specific timing arrangements as defined by a treatment profile which is configured so that the emitted electromagnetic energy stimulates and/or inhibits an immune response in a host when the emitted electromagnetic energy is delivered to the host via the electromagnetic energy applicator.

3. The electromagnetic energy delivery system as claimed in claim 2, wherein at least one of:
the controlled electromagnetic energy source is configured to emit electromagnetic energy according to a treatment profile which is configured so that the emitted electromagnetic energy stimulates and/or inhibits an immune response in the host without causing necrosis of cells or tissue of the host when the emitted electromagnetic energy is delivered to the host via the electromagnetic energy applicator;
wherein the controlled electromagnetic energy source is configured to emit electromagnetic energy according to a treatment profile which is configured so that the emitted electromagnetic energy heats cells or tissue of the host when the emitted electromagnetic energy is delivered to the host via the electromagnetic energy applicator;
wherein the controlled electromagnetic energy source is configured to emit electromagnetic energy according to a treatment profile which is configured so that the emitted electromagnetic energy heats cells or tissue of the host without ablating the cells or tissue of the host when the emitted electromagnetic energy is delivered to the host via the electromagnetic energy applicator.

4. (canceled)

5. (canceled)

6. The electromagnetic energy delivery system as claimed in claim 2, wherein:
the treatment profile defines one or more delivery periods and the controlled electromagnetic energy source is configured to only emit electromagnetic energy only during each delivery period, and optionally
the treatment profile defines one or more series of pulses, each pulse having a duration equal to the delivery period, and optionally
each delivery period is one thousandth of a second, one second, two seconds, two to three seconds, or any time duration up to twenty seconds or up to one or two or ten minutes, and optionally
the treatment profile defines one, five to ten, up to one hundred or one thousand delivery periods, and optionally
the treatment profile defines a plurality of delivery periods configured so that the emitted electromagnetic energy is pulsed at a rate of 5 Hz.

7. The electromagnetic energy delivery system as claimed in claim 2, wherein at least one of:
the controlled electromagnetic energy source is configured to emit electromagnetic energy according to a treatment profile which is configured so that the emitted electromagnetic energy is emitted with a power in the range 1-50 W, such as 8 W-10 W, 2 W-5 W or 3 W-8 W;
the controlled electromagnetic energy source is configured to emit electromagnetic energy at a microwave frequency, for example wherein the controlled electromagnetic energy source is configured to emit electromagnetic energy at a frequency in a frequency range of 1-300 GHz;
the controlled electromagnetic energy source is configured to emit electromagnetic energy according to a treatment profile which is configured so that the emitted electromagnetic energy is amplitude modulated, for example at a frequency in the range 1-100 KHz;
the controlled electromagnetic energy source is configured to emit electromagnetic energy according to a treatment profile which is configured so that the emitted electromagnetic energy is modulated according to a pulse width modulation (PWM) or an on/off keying (OOK) modulation scheme;
the controlled electromagnetic energy source is configured to emit electromagnetic energy according to a treatment profile which is configured so that the emitted electromagnetic energy is frequency modulated, for example at a frequency in the range 1-100 KHz.

8. (canceled)

9. (canceled)

10. (canceled)

11. (canceled)

12. An electromagnetic energy delivery system as claimed in claim 2, wherein the controlled electromagnetic energy source is configured to receive one or more signals from one of more physiological parameter sensors and to adapt the treatment profile according to the one or more received signals and, optionally,
wherein the controlled electromagnetic energy source is configured to receive a PQRST heart rhythm signal from an ECG sensor and to adapt the treatment profile according to the PQRST heart rhythm cycle signal, for example by dynamically synchronising the treatment profile to correspond with a particular point in the PQRST heart rhythm cycle and, optionally,
wherein the controlled electromagnetic energy source is configured to receive a neural oscillation signal from an EEG sensor and to adapt the treatment profile according to the neural oscillation signal, for example by dynamically synchronising the treatment profile to correspond with the neural oscillation signal and, optionally,
wherein the controlled electromagnetic energy source is configured to receive a blood pressure signal from a blood pressure sensor and to adapt the treatment profile according to the blood pressure signal, for example by dynamically synchronising the treatment profile to correspond with the blood pressure signal.

13. (canceled)

14. (canceled)

15. (canceled)

16. A method of electromagnetic energy delivery, comprising delivering energy in specific timing arrangements to improve an immune response.

17. The method of electromagnetic energy delivery as claimed in claim 16, comprising:
emitting electromagnetic energy according to the specific timing arrangements as defined by a treatment profile; and
delivering the emitted electromagnetic energy to the host,
wherein the treatment profile is configured so that the emitted electromagnetic energy stimulates and/or inhibits the immune response in the host when the emitted electromagnetic energy is delivered to the host.

18. The method of electromagnetic energy delivery as claimed in claim 17, comprising at least one of:
emitting electromagnetic energy according to a treatment profile which is configured so that the emitted electromagnetic energy stimulates and/or inhibits an immune response in the host without causing necrosis of cells or tissue of the host when the emitted electromagnetic energy is delivered to the host;
emitting electromagnetic energy according to a treatment profile which is configured so that the emitted electromagnetic energy heats cells or tissue of the host when the emitted electromagnetic energy is delivered to the host;
emitting electromagnetic energy according to a treatment profile which is configured so that the emitted electromagnetic energy heats cells or tissue of the host without ablating the cells or tissue of the host when the emitted electromagnetic energy is delivered to the host.

19. (canceled)

20. (canceled)

21. The method of electromagnetic energy delivery as claimed in claim 17, wherein:
the treatment profile defines one or more delivery periods and the method comprises only emitting electromagnetic energy during each delivery period, and optionally
the treatment profile defines one or more series of pulses, each pulse having a duration equal to the delivery period, and optionally
each delivery period is one thousandth of a second, one second, two seconds, two to three seconds, or any time duration up to twenty seconds or up to one or two or ten minutes, and optionally
the treatment profile defines one, five to ten, up to one hundred or one thousand delivery periods, and optionally
the treatment profile defines a plurality of delivery periods configured so that the emitted electromagnetic energy is pulsed at a rate of 5 Hz, and optionally
the treatment profile defines a schedule of treatments, each treatment comprising at least one delivery period, and each treatment being separated by an interval of one or more weeks, for example four weeks, and optionally
the treatment profile defines one or more further delivery periods after a follow-up interval of at least 12 weeks.

22. The method of electromagnetic energy delivery as claimed in claim 17, comprising at least one of:
emitting electromagnetic energy according to a treatment profile which is configured so that the emitted electromagnetic energy is emitted with a power in the range 1-50 W, such as 8 W-10 W, 2 W-5 W or 3 W-8 W;
emitting electromagnetic energy at a microwave frequency, for example at a frequency in the range 1-300 GHz;
emitting electromagnetic energy according to a treatment profile which is configured so that the emitted electromagnetic energy is amplitude modulated for example at a frequency in the range 1-100 KHz;
emitting electromagnetic energy according to a treatment profile which is configured so that the emitted electromagnetic energy is frequency modulated for example at a frequency in the range 1-100 KHz.

23. (canceled)

24. (canceled)

25. (canceled)

26. The method of electromagnetic energy delivery as claimed in claim 17, comprising:
receiving one or more signals from one of more physiological parameter sensors; and
adapting the treatment profile according to the one or more received signals.

27. The method of electromagnetic energy delivery as claimed in claim 26, comprising:
receiving a PQRST heart rhythm signal from an ECG sensor; and
adapting the treatment profile according to the PQRST heart rhythm cycle signal, for example by dynamically synchronising the treatment profile to correspond with a particular point in the PQRST heart rhythm cycle.

28. The method of electromagnetic energy delivery as claimed in claim 26, comprising:
receiving a neural oscillation signal from an EEG sensor; and
adapting the treatment profile according to the neural oscillation signal, for example by dynamically synchronising the treatment profile to correspond with the neural oscillation signal.

29. The method of electromagnetic energy delivery as claimed in claim 26, comprising:
receiving a blood pressure signal from a blood pressure sensor; and
adapting the treatment profile according to the blood pressure signal, for example by dynamically synchronising the treatment profile to correspond with the blood pressure signal.

30. The method of electromagnetic energy delivery as claimed in claim 17, comprising adapting the treatment profile according to a measurement representative of an immune cycle of the host.

31. The method of electromagnetic energy delivery as claimed in claim 30, wherein the measurement representative of the immune cycle of the host comprises at least one of measurement of C-reactive protein (CRP) levels in the blood, T-regulatory cells (low state) and T-effector cells (high states).

32. The method of electromagnetic energy delivery as claimed in claim 17, comprising administering one or more other therapies to the host, before, during and/or after emitting electromagnetic energy according to the treatment profile and delivering the electromagnetic energy to the host.

33. The method of electromagnetic energy delivery as claimed in claim 32, wherein the one or more other therapies comprise at least one of: radiotherapy, chemotherapy, immunotherapy, traditional pharmacological therapies, transcutaneous electrical nerve stimulation (TENS) and frequency rhythmic electrical modulation systems (FREMS).

34. A treatment profile for use with the electromagnetic energy delivery system as claimed in claim 2 or the method of electromagnetic energy delivery as claimed in claim 17.
